(19) United States
(12) Plant Patent Application Publication
(10) Pub. No.: US 2004/0010831 P1
(43) Pub. Date: Jan. 15, 2004

(54) Geranium plant named 'Olivia'

(76) Inventor: Laura B. McCarthy Bartlett, Sudbury, MA (US)

Correspondence Address: J. P. Bartlett Co., Inc., Laura B. McCarthy, President, 578 Boston Post Rd., Route 20, Sudbury, MA, 01776, US

(21) Appl. No.: 10/195,151

(22) Filed: Jul. 15, 2002

(51) Int. Cl. A01H 5/00

(52) U.S. Cl. PLT/328

(57) ABSTRACT

This invention is a new and distinct cultivar of a zonal geranium (Pelagronium×hortorum) named Olivia. With a brilliant array of colors within the plant's large, full, early blossoming flowers and dark zoned leaves, ‘Olivia’ is unique stable geranium.

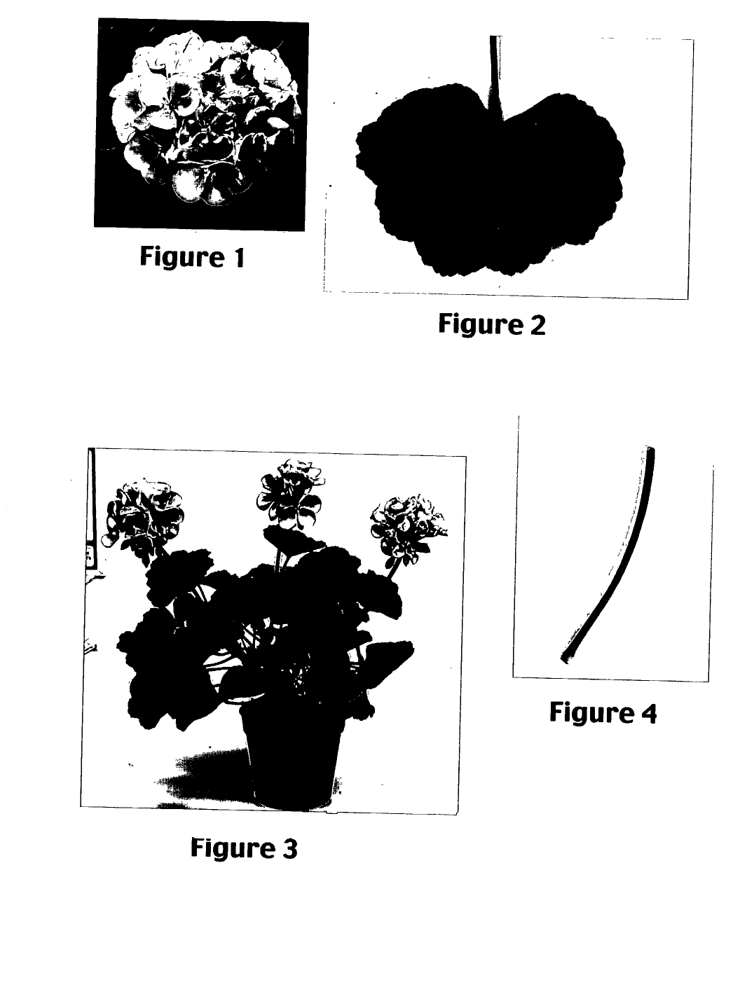

### BACKGROUND OF THE INVENTION

‘Olivia’ is one of J. P. Bartlett's original geranium varieties. It has been sold as a wholesale product for numerous years. ‘Olivia’ belongs to the genus Pelargonium and the species Zonal hortorum. ‘Olivia’ has been marketed to retail greenhouses, garden centers, and landscape companies over the years.

### DESCRIPTION

The cultivar was developed from and organized scientifically by a designed breeding program conducted at the J.P. Bartlett Company, Incorporated.; 578 Boston Post Road; Sudbury, Mass.; United States of America 01776. The breeding program combined cultivars from it's past breeding with other American cultivars.

The breeders at J.P. Bartlett Co., Inc. used ‘Pink Expectation’ and a trial variety; number ‘1192,’ in a cultivated area to create ‘Olivia’. ‘Pink Expectation’ was used as the male plant in this experiment. This flower is a medium salmon color with just a hint of pink. The leaf has a dark green zoning. ‘Pink Expectation’ has a medium growth habit compared to that of the female plant, trial number ‘1192’. This was more of an experimental plant, as it was never used directly for profit. The flower of ‘1192’ has more of a light salmon color with a touch of pink to accent the blossom. The leaf was identified as having very light zoning. ‘Olivia's’ plant habit is symmetrical with excellent basal as well as a lateral branching habit. By using the male pollen from ‘Pink Expectation’ to pollinate the female ‘1192’. J.P. Bartlett Co., Inc came up with the trial number ‘1532’. For three years trial number ‘1532’ was carefully watched. After trial number ‘1532’ proved that it was a stable and true variety, it was given the name ‘Olivia’. Since ‘Olivia’ has been created there have been no known sports or any other variation of this geranium. ‘Olivia’ has always remained true to the color, leaf, and overall growth of the plant.

### SUMMARY

This invention is one of the many traditional and unique cultivated geraniums; Pelargonium×hortorum, J.P. Bartlett Co., Inc. has to offer. The variety has adapted very well to both greenhouse growth and garden performance. However, under the cultivated area ‘Olivia's’ growth performance excels. Olivia is a large plant with a dense mounded habit. This along with a hi vigor outperforms much of the competition. Excellent floriferous flowering and brilliant color makes ‘Olivia’ enjoyable throughout all of the seasons. The huge blossoms make ‘Olivia’ a hard plant to miss. The average measured size of a blossom in full sun is between one hundred and one hundred and twenty millimeters. The plant itself is quite large, but it never diminishes how full a plant ‘Olivia’ is. On average the plant itself can get over 457.2 millimeters tall, not including the stem or flower. The free branching nature of ‘Olivia’ allows the plants girth to grow and flourish. The width of a single plant is measured to be 762 millimeters and growing. The multiple shades of color in the plant separates itself from any other. The semi-double blossom is a medium coral streaking to a pale coral edge. ‘Olivia's’ stem is a distinguished bronze, while the leaf produces a unique variation of colors all in its own. ‘Olivia's’ standout performance is regarded highly due its excellent growth habit, size, blossom and leaf.

### DRAWING

Accompanying this application is a photographed picture of the plant and its aspects.

Figure one shows the large, semi-double blossom and the coral color described in the summary.

Figure two enables us to see the deep green leaf with the darker green zone.

Figure three shows the overall plant, this is a single plant from a cutting in a four inch pot. The height, width and growth habit are clearly seen here.

Figure four depicts the stem. The strong bronze color runs through the stem making it one of the unique aspects of the plant. The color of the blossom, leaf and stem are as true as reasonably possible.

### BOTANICAL DESCRIPTION

The cultivar ‘Olivia’ belongs to the Pelargonium×hortorum genus and species. This variety was bred by cross pollination to other varieties of geraniums. The parent plant's of ‘Olivia’ are ‘Pink Expectation’ and trial number ‘1192’, described above. The growth habit of this plant is medium to vigorous. It is a very large, full plant that performs well in both a greenhouse and in a natural environment such as a garden. It will grow more compact under drier conditions but has the ability to be a large and vigorous plant with sufficient water and partial shade. This variety performs remarkably when bearing the natural elements. The blossoms are tolerant to rain, not turning brown and rotting after a clay or so of straight rain. ‘Olivia’ buds early and is consistent with a larger and longer lasting blossom than many other cultivars. During the winter ‘Olivia’ has survived in the greenhouse where the temperature is maintained above 50° F. ‘Olivia’ is produced by cuttings from the stock plants. With excellent rooting quality it takes approximately ten to twelve weeks (depending on climate) for the cutting to mature into a rooted, full plant. The amount of cuttings a single plant can produce in a six month period is sixteen give or take the climate and demand of product.

1. J.P. Bartlett Co., Inc has bred an excellent variety of Pelargonium×hortorum that excels in cultivated areas and garden performance, showing off the beauty of the floriferous blossoms, the dark zone leaf, and the large, vigorous, growing habit of ‘Olivia.’
